(19) United States
(12) Patent Application Publication
(10) Pub. No.: US 2026/0229381 A1
(43) Pub. Date: Aug. 6, 2026

(54) COLLIMATORS FOR ULTRA-HIGH DOSE RATE RADIATION

(71) Applicant: Ohio State Innovation Foundation, Columbus, OH (US)

(72) Inventors: Ashley Joy CETNAR, Columbus, OH (US); Sagarika JAIN, Columbus, OH (US); Nilendu GUPTA, Columbus, OH (US); Ahmet Sedat AYAN, Columbus, OH (US); Jeffrey WOOLLARD, Columbus, OH (US); Gavin GRAEPER, Columbus, OH (US)

(21) Appl. No.: 19/152,065

(22) PCT Filed: Jan. 30, 2024
(86) PCT No.: PCT/US2024/013494

§ 371 Date: Jul. 30, 2025

Related U.S. Application Data
(60) Provisional application No. 63/482,175, filed on Jan. 30, 2023.

(51) Int. Cl. G21K 1/02; A61N 5/10; B22F 10/80; B29C 64/386; B33Y 50/00; B33Y 80/00

(52) U.S. Cl. CPC G21K 1/02; A61N 5/1045; B22F 10/80; B29C 64/386; B33Y 50/00; B33Y 80/00; A61N 2005/1089; B22F 2301/052; B22F 2301/10

(57) ABSTRACT

A collimator assembly for a linear accelerator for use in FLASH radiotherapy includes a collimator and a tray. The collimator includes an elongated body and a base and can also include an opening that defines a central bore formed in the elongated body and the base and extending the length of the collimator along a central axis. The tray includes a central opening having a larger cross-section than the elongated body of the collimator but smaller than the base of the collimator to allow only the elongated body of the collimator to pass therethrough such that the collimator is retained by the tray for reversible installation on the linear accelerator. The tray is adapted to be removably installed on accessory rails of the linear accelerator. Both the collimator and tray may be fabricated by 3D printing to allow for customization.

### CROSS-REFERENCE TO RELATED APPLICATIONS

This application claims the benefit of, and priority to, U.S. Provisional Patent Application No. 63/482,175, filed Jan. 30, 2023, which is incorporated herein by reference in its entirety.

### BACKGROUND

FLASH radiotherapy (FLASH-RT) is an emerging technology in the field of radiation therapy, e.g., for the treatment of tumors, involving the delivery of ultra-high dose rate radiation to a target (e.g., tissue). In FLASH-RT, a specialized linear accelerator is used to irradiate target areas on a subject at an ultra-high dose rate (e.g., 40 Gy/s as compared to 0.5-5 Gy/min in conventional radiotherapy). Unlike conventional radiotherapy, FLASH-RT has been demonstrated to limit trauma to “normal” tissue around a tumor without losing effectiveness on the tumor itself. In this emerging field, there is a lack of standardization in the development and implementation of devices and components for ultra-high dose rate linear accelerators, such as collimation systems. Generally, collimation systems are not provided with commercially available linear accelerators and, even if they are available, are highly proprietary and not suitable for use on more than one type of machine. Traditional collimation devices may also be unsuitable for the ultra-high dose rates associated with FLASH-RT.

### SUMMARY

One implementation of the present disclosure is a collimator assembly for a linear accelerator for use in ultra-high dose rate (FLASH) radiotherapy, the collimator assembly including: a collimator including an elongated body and a base, wherein the elongated body is cylindrically shaped, wherein the base is cylindrically shaped or disk-shaped and is positioned at one end of the elongated body, wherein the elongated body is a first diameter and the base is a second diameter that is greater than the first diameter, and wherein an opening that defines a central bore is formed in the elongated body and the base, the opening extending a length of the collimator along a central axis; and a tray including a first side, a second side opposite the first side, and a central opening that extends from the first side to the second side, wherein the tray is adapted to be removably installed on accessory rails of the linear accelerator, wherein the central opening of the tray is a third diameter that greater than the first diameter of the elongated body of the collimator but smaller than the second diameter of the base of the collimator to allow only the elongated body of the collimator to pass therethrough such that the base of the collimator interfaces with the first side of the tray, thereby retaining the collimator for reversible installation on the linear accelerator.

Another implementation of the present disclosure is a method of fabricating a collimation system for a linear accelerator, for use in ultra-high dose rate (FLASH) radiotherapy, the method including: obtaining, by a first computing device, from a remote computing device, 3D printing files or 3D models of a collimator and a collimator tray, wherein the collimator tray is configured to retain the collimator for reversible installation onto the linear accelerator; operating, by the first computing device, a 3D printer to print the collimator, wherein the collimator includes an elongated body and a base, wherein the base is wider than the elongated body, and wherein an opening that defines a central bore is formed in the elongated body and the base, the opening extending a length of the collimator along a central axis; and operating, by the first computing device, the 3D printer to print the collimator tray, wherein the collimator tray includes a first side, a second side opposite the first side, and a central opening that extends from the first side to the second side, wherein the central opening is sized to allow only the elongated body of the collimator, but not the base of the collimator, to pass therethrough, and wherein the tray is adapted to be removably installed on accessory rails of the linear accelerator; wherein the collimation system is assembled by inserting the elongated body of the collimator into the central opening of the tray such that the base of the collimator interfaces with the first side of the tray, and wherein the collimation system is installed onto the linear accelerator such that the collimator is positioned at an exit window of the linear accelerator.

Yet another implementation of the present disclosure is a method of operating a linear accelerator configured for ultra-high dose rate (FLASH) radiotherapy, the method including: providing a collimator including an elongated body and a base, wherein the elongated body is cylindrically shaped, wherein the base is cylindrically shaped or disk-shaped and is positioned at one end of the elongated body, wherein the elongated body is a first diameter and the base is a second diameter that is greater than the first diameter, and wherein an opening that defines a central bore is formed in the elongated body and the base, the opening extending a length of the collimator along a central axis; and providing a tray including a first side, a second side opposite the first side, and a central opening that extends from the first side to the second side, wherein the tray is adapted to be removably installed on accessory rails of the linear accelerator; inserting the elongated body of the collimator into the central opening of the tray, wherein the central opening of the tray is a third diameter that greater than the first diameter of the elongated body of the collimator but smaller than the second diameter of the based on the collimator to allow only the elongated body of the collimator to pass therethrough such that the base of the collimator interfaces with the first side of the tray; and installing the tray on accessory rails of the linear accelerator such that the collimator is positioned at an exit window of the linear accelerator.

Additional advantages will be set forth in part in the description that follows or may be learned by practice. The advantages will be realized and attained by means of the elements and combinations particularly pointed out in the appended claims. It is to be understood that both the foregoing general description and the following detailed description are exemplary and explanatory only and are not restrictive, as claimed.

### BRIEF DESCRIPTION OF THE DRAWINGS

FIG. 1 is a diagram showing a side view of an example linear accelerator with a collimation system installed, according to some implementations.

FIG. 2 is a diagram showing a front view of the example linear accelerator and collimation system of FIG. 1, according to some implementations.

FIG. 3 is a diagram showing a bottom-up view of the example linear accelerator and collimation system of FIG. 1, according to some implementations.

FIG. 4 is a side view of a collimator of the disclosed collimation system, according to some implementations.

FIG. 5 is a side perspective view of the collimator of FIG. 4, according to some implementations.

FIG. 6 is a bottom perspective view of the collimator of FIG. 4, according to some implementations.

FIG. 7 is a perspective view of an accessory tray of the disclosed collimation system, according to some implementations.

FIG. 8 is a side view of the accessory tray of FIG. 7, according to some implementations.

FIG. 9 is a top-down view of an accessory tray of FIG. 7, according to some implementations.

FIG. 10A is a side view of the disclosed collimation system, according to some implementations.

FIG. 10B is a top-down view of the disclosed collimation system, according to some implementations.

FIG. 11 is a block diagram of a system for fabricating a collimation system including a collimator and a tray, according to some implementations.

FIG. 12 is a flow chart of a process for fabricating and using a collimation system including a collimator and a tray, according to some implementations.

FIGS. 13-15 are various perspective views of a prototype of the disclosed collimation system installed on an example linear accelerator, according to some implementations.

FIG. 16 is an example radiotherapy dose applied to a radiochromic film using the disclosed collimation system, according to some implementations.

FIG. 17 is a diagram showing another example radiotherapy dose applied to a radiochromic film using the disclosed collimation system, according to some implementations.

FIG. 18 is a graph showing a path profile of a radiotherapy dose applied to dosimetrically water equivalent plastic for testing, according to some implementations.

FIG. 19 is a graph showing a percent depth dose curve using radiochromic film in water for testing, according to some implementations.

FIGS. 20A and 20B are graphs of dose profiles when using the disclosed collimation system, according to some implementations.

Various objects, aspects, features, and advantages of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements.

### DETAILED DESCRIPTION

Referring generally to the figures, a collimation system for use with ultra-high dose rate capable linear accelerators, e.g., for FLASH-RT, along with corresponding methods of fabrication and use, are shown, according to various implementations. Collimation systems are standard for electron delivery from therapeutic radiation-generating devices (e.g., linear accelerators or “linacs”). In some cases, primary and secondary collimation is provided within the head of a clinical linac, and tertiary collimation is provided externally to shape the field closer to the patient. However, as mentioned above, collimation systems are generally not suited for, or provided with, commercially available linacs that are capable of the ultra-high dose rates associated with FLASH-RT.

The collimation system described herein generally includes a collimator, which is a device that narrows or focuses beams particles or waves (e.g., the output of a linear accelerator), and a tray adapted to hold the collimator. More specifically, the tray is designed to retain the collimator for removable (e.g., non-permanent) installation onto a clinical linac. The collimation system described herein is generally configured to minimize the distance between the collimator and an exit window of a linac in order to increase dose rate, while still providing clinically acceptable collimation for electron beam dosimetry. Notably, the collimator and/or tray can be adapted to fit many common types of clinical linacs used for FLASH radiotherapy- and, indeed, can also be used for more conventional radiotherapy, e.g. at lower dose rates. Additionally, the size and/or shape of the collimator can be customized based on the desired field size of irradiation.

### Linacs for FLASH-RT

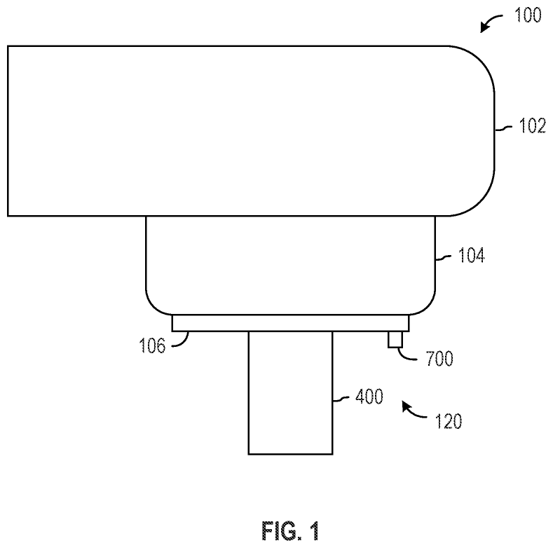

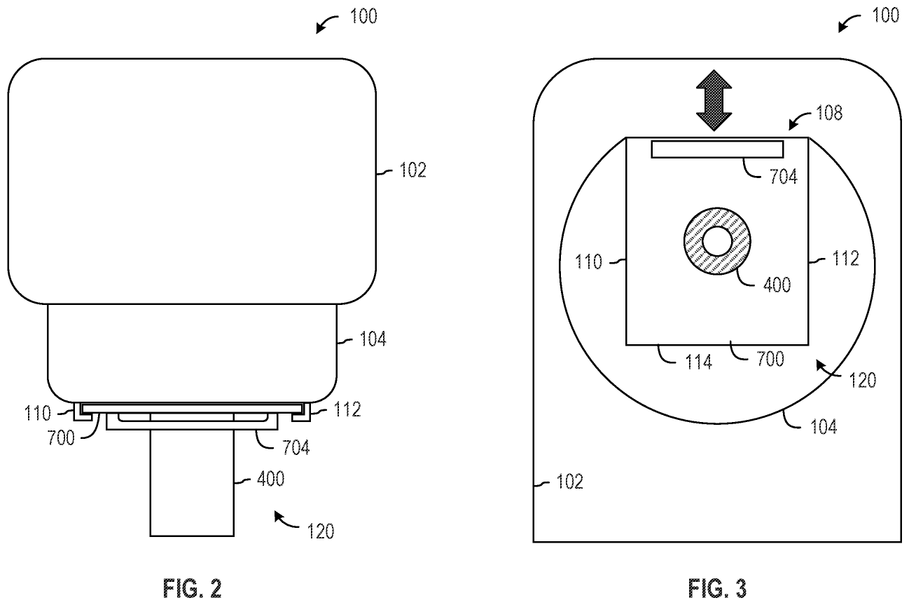

Turning first to FIGS. 1-3, a portion of an example linear accelerator (linac) 100 is shown from a variety of perspectives (e.g., FIG. 1 is a side view, FIG. 2 is a front-side view, and FIG. 3 is a bottom-up view). As described herein, linac 100 is generally a clinical linac configured to deliver the ultra-high dose rate radiation (e.g., upwards of 40 Gy/s) associated with FLASH-RT; therefore, linac 100 may be considered “FLASH-RT” enabled or “FLASH-RT” capable. In some implementations, linac 100 is able to provide an electron beam energy of about 16 megaelectron volts (MeV).

Linac 100 generally includes a body 102, also called a gantry, and a treatment head 104. For conciseness, body 102 and treatment head 104, along with operating procedures for linac 100, are not described in detail herein, as they will be readily understood by those of skill in the art. However, it should be understood that treatment head 104 generally includes an exit window (not illustrated) for emitting radiation. Fixed to a bottom side of treatment head 104 is an accessory rail assembly 106 used to attach various accessories at or near an exit window of treatment head 104. As shown in FIG. 3, for example, accessory rail assembly 106 of linac 100 is generally circular in shape (e.g., when viewed from the bottom) with an opening 108 for inserting accessories. Opening 108 is generally defined by first and second side rails 110, 112 for retaining collimator assembly 120 and an end wall 114. With additional reference to FIG. 2, in some implementations, first and second side rails 110, 112 are L-shaped when viewed from the front. However, it should be understood that the accessory rail or accessory mounting points on various linacs may vary in size, shape, arrangement, and configuration, all of which are contemplated herein.

As shown, a collimation system 120 is installed onto linac 100, e.g., covering the exit window, using accessory rail assembly 106. Collimation system 120, as described herein, generally includes two main components: a collimator 400 and a collimator tray 700, both described in greater detail below. Collimator tray 700 is generally configured to retain collimator 400 and is used to attach collimator 400 to linac 400. As shown in FIGS. 2 and 3, for example, collimator tray 700 may have a generally square or rectangular shape corresponding to accessory rail assembly 106; however, it should be appreciated that collimator tray 700 may be another shape (e.g., circular or disk-shaped) based on the configuration of accessory rail assembly 106. In some implementations, the thickness of collimator tray 700 is selected based on the configuration of accessory rail assembly 106, or more specifically, based on the size of first and second side rails 110, 112. In some implementations, collimator tray 700 includes a handle 704 to facilitate insertion/removal of collimator assembly 120 on accessory rail assembly 106. For example, as illustrated in FIG. 3, collimator assembly 120 is inserted or removed by grasping handle 704 and sliding collimator assembly 120 into or out of opening 108.

### Example Collimator

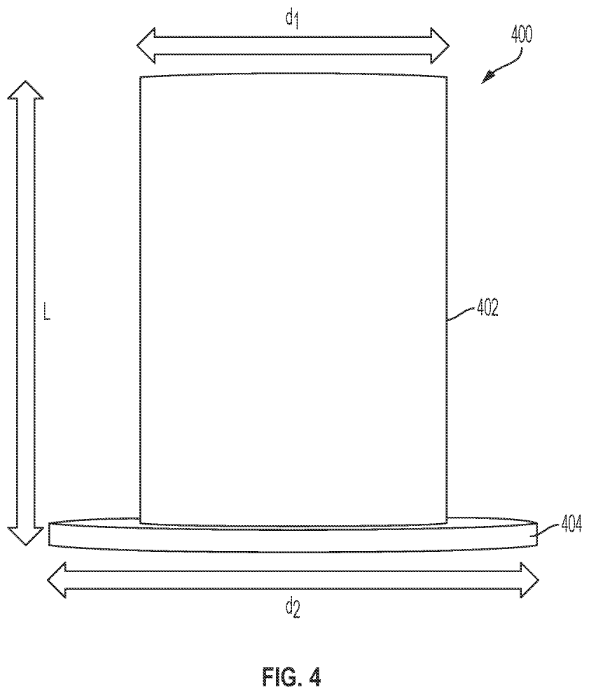

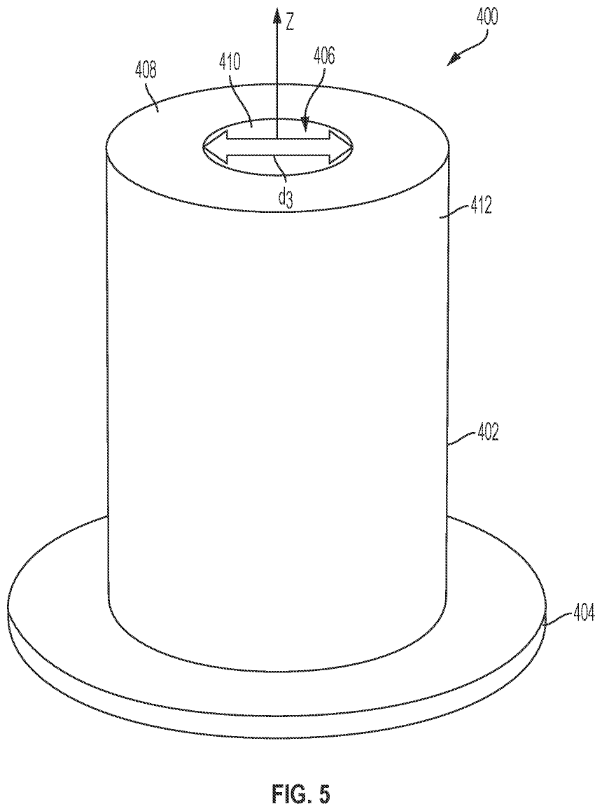

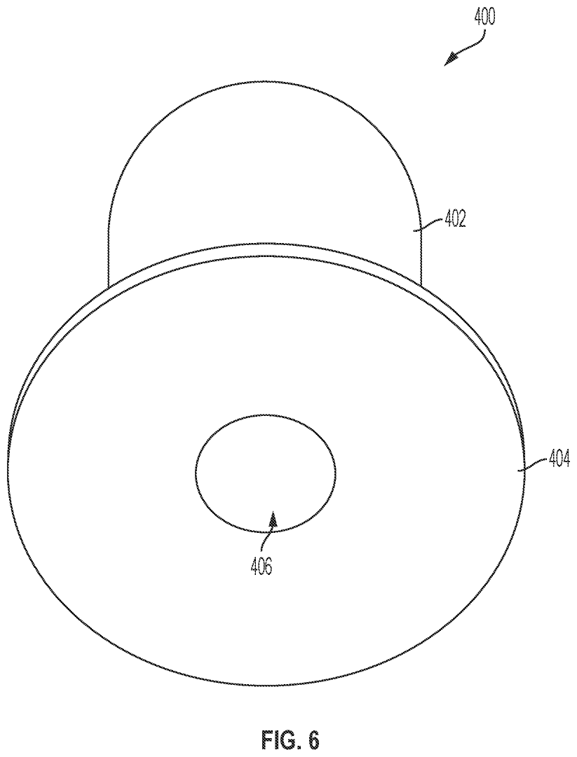

Referring now to FIGS. 4-6, one example implementation of collimator 400 is shown in greater detail. In particular, FIG. 4 shows a side-view of collimator 400; FIG. 5 shows a side-perspective view of collimator 400; and FIG. 6 shows a bottom-perspective view of collimator 400, according to various implementations. As mentioned above, collimator 400 is a device that narrows or focuses the output of linac 100 to target a treatment site (e.g., a tumor). Notably, the specific size and shape of collimator 400, as described herein, can be customized or modified based on the desired resulting field size or shape and/or based on the particular configuration of the linac that collimator 400 is being used on. Accordingly, it should be appreciated that the following description of collimator 400 is not intended to be limiting and that other configurations of collimator 400 are contemplated herein.

As shown, collimator 400 is defined by a longitudinal body 402 and a base 404. Generally, longitudinal body 402 and base 404 are integrally formed; however, this disclosure also contemplates implementations in which longitudinal body 402 and base 404 are separately formed and subsequently fixedly attached (e.g., using an adhesive, by welding, etc.). Both longitudinal body 402 and base 404 are shown to be generally cylindrical in shape (e.g., with base 404 being disk-shaped), in the illustrated implementation; however, it should be appreciated that one or both of longitudinal body 402 and base 404 could be formed in another shape based on the specific use case. For example, in some implementations, longitudinal body 402 may be rectangular, square-shaped, or non-uniform in shape.

Generally, however, base 404 is larger in size than longitudinal body 402. In the implementation shown, base 404 has a larger diameter (d₂) than longitudinal body 402. In some implementations, the diameter (d₁) of longitudinal body 402 is 3 centimeters (cm); however, in other implementations, the diameter (d₁) of longitudinal body 402 can be greater than or less than 3 cm. Accordingly, in some such implementations, the diameter (d₂) of base 404 is greater than 3 cm. In some implementations, the diameter (d₂) of base 404 is between 10% and 50% greater than the diameter (d₁) of longitudinal body 402. Generally, however, the diameter (d₁) of longitudinal body 402 can be selected based on the desired field size of irradiation.

Turning to FIGS. 5 and 6, collimator 400 is shown to further include a central bore 406 (also referred to as a central lumen) which extends the length of collimator 400, e.g., along a central axis (z) of collimator 400. Put another way, central bore 406 is a hollow opening that extends through the center of, and the length of, longitudinal body 402 and base 404. In some implementations, central bore 406 is generally cylindrical in shape (e.g., as shown in FIGS. 5 and 6). In some such implementations, central bore 406 is defined by an inner surface 410 of a side wall 408 of longitudinal body 402. Side wall 408 may further include an outer surface 412 which defines an exterior surface of collimator 400. Central bore 406 may be a third diameter (d₃) that is smaller than the diameter (d₁) of longitudinal body 402.

While shown in FIGS. 5 and 6 as being generally cylindrical, it should be appreciated that the size and/or shape of central bore 406 can be selected or modified based on the particular use case of collimator 400. For example, the diameter (d₃) of central bore 406 may be selected based on the desired resulting field size of irradiation. In some implementations, central bore 406 can have a tapered shape, e.g., such that central bore 406 has a varying diameter along the length of collimator 400, to match the divergence of the beam. In some implementations, collimator 400 does not include central bore 406 (e.g., longitudinal body 402 and base 404 are solid). It should also be appreciated that, in some implementations, central bore 406 is a shape other than cylindrical. For example, central bore 406 may have a square or rectangular cross-section, a non-uniform cross-section, etc. In this way, the shape and size of central bore 406 can be selected or customized based on the desired implementation of collimator 400 (e.g., to customize the size and shape of the field of irradiation).

In some implementations, as described below in greater detail, collimator 400 is formed using three-dimensional (3D) printing. In some such implementations, collimator 400 is fabricated using a material having a similar density to water, such as a material having a density between 0.9 and 1.1g/cm³. Example materials include acrylonitrile butadiene styrene (ABS) or polylactic acid (PLA). Notably, low-Z materials (e.g., PLA) are less prone to activation than high atomic number (Z) or “high-Z” materials, as discussed in greater detail below. In other implementations, collimator 400 is 3D printed using higher-density materials such as aluminum or copper alloys or other metals. In yet other implementations, collimator 400 is formed by molding, casting, machining, or any other suitable techniques. In general, the density of the material used to manufacture collimator 400 determines the overall length (L) of collimator 400 based on the nominal energy of the radiation beam to be produced. For example, configured for a linac that produces 16 MeV electrons, collimator 400 may have a length (L) of at least 90 millimeters (mm). With higher-density materials such as copper or aluminum alloys, the length (L) of collimator 400 may be reduced to 10-35 mm to further maximize the dose rate. Collimators (e.g., collimator 400) of various diameters can be printed or otherwise formed depending upon the desired field size of irradiation.

### Collimator (Accessory) Tray

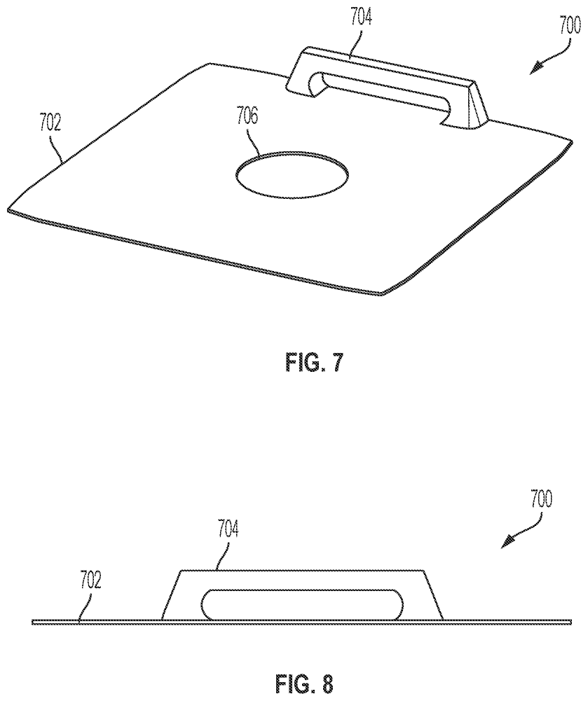

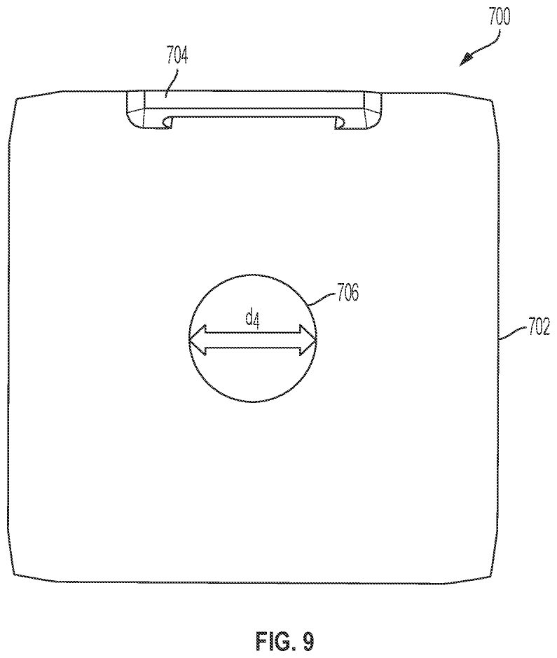

Referring now to FIGS. 7-9, collimator tray 700 is shown in greater detail. In particular, FIG. 7 shows a perspective view of collimator tray 700; FIG. 8 shows a side perspective view of collimator tray 700; and FIG. 9 shows a top-down view of collimator tray 700, according to various implementations. As described above, collimator tray 700 (or simply tray 700) is generally designed to be placed outside of the exit window of a linac (e.g., linac 100) in order to retain collimator 400 at or near the exit window. Specifically, collimator tray 700 may be sized to engage with accessory rail assembly 106 for installation/removal. To this point, the dimensions of collimator tray 700 may be selected or customized based on the requirements (e.g., accessory rail configuration and/or size) of the linac on which collimation system 120 is to be installed.

In the example shown, collimator tray 700 is generally square in shape, having four equal-length side edges that define a body 702 of the tray which corresponds to opening 108 of accessory rail assembly 106 of linac 100. In other implementations, collimator tray 700 may be rectangular in shape and/or may be otherwise sized to fit the accessory rails or accessory attachment system of other linacs. Collimator tray 700 may also include handle 704 to facilitate the insertion/removal of collimation system 120 into accessory rail assembly 106. As shown, handle 704 may be integrally formed into collimator tray 700. Alternatively, in some implementations, handle 704 is separately formed and then fixedly attached to collimator tray 700 (e.g., using an adhesive). In any case, handle 704 may extend outward from one side (e.g., the bottom side) of collimator tray 700 and may be positioned along one side edge of collimator tray 700.

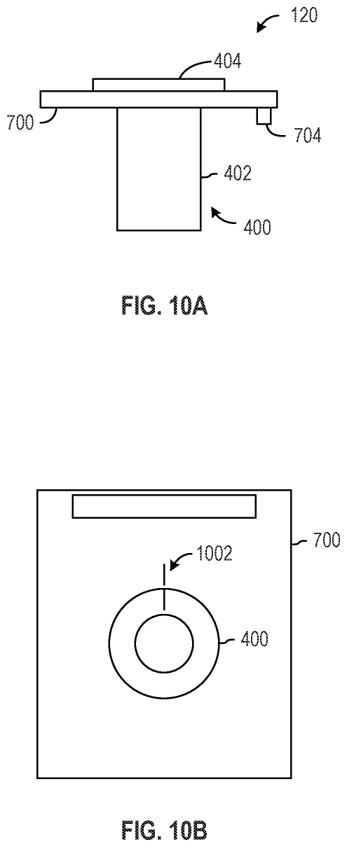

Collimator tray 700 is shown to further include a central opening 706 which is formed into body 702. Central opening 706 generally extends between first and second (e.g., top and bottom) sides of collimator tray 700 to form an opening (e.g., a hole) through body 702 of collimator tray 700. With additional reference to FIG. 10, central opening 706 is configured to allow longitudinal body 402 of collimator 400 to pass therethrough such that, when collimator 400 is installed into collimator tray 700, longitudinal body 402 extends away from one side of collimator tray 700 (e.g., the second side, if collimator 400 is inserted from the first side of collimator tray 700). Specifically, in implementations where longitudinal body 402 of collimator 400 is cylindrical in shape, the diameter (d₄) of central opening 706 may be selected such that only longitudinal body 402 of collimator 400 can pass therethrough. In other words, the diameter (d₄) of central opening 706 may be slightly greater than the diameter (d₁) of longitudinal body 402 but smaller than the diameter (d₂) of base 404. In this way, base 404 of collimator 400 retains collimator 400 when installed onto collimator tray 700, as shown in FIG. 10A.

To assemble collimation system 120, e.g., for installation on a linac, collimator 400 is first installed in collimator tray 700 by inserting longitudinal body 402 of collimator 400 into central opening 706. As noted above, base 404 of collimator 400 prevents collimator 400 from passing completely through central opening 706. Together, collimator 400 and collimator tray 700 form collimator assembly 120, which can then be reversibly or removably installed onto a linac by, for example, sliding collimator tray 700 onto accessory rail assembly 106 (e.g., using handle 704).

In some implementations, collimator 400 and collimator tray 700 include corresponding alignment elements for, e.g., rotationally aligning the two components. For example, in some cases, as described above, central bore 406 of collimator 400 is non-uniform; therefore, alignment with collimator tray 700 may be beneficial to inform a user of the size and positioning of the resultant field of irradiation. Additionally, alignment elements may help to ensure that collimator 400 is positioned in the same way each time it is installed on linac 100. In some implementations, as illustrated in FIG. 10B, these alignment elements may include corresponding markings 1002 (e.g., a line, dots, etc.) on each of collimator 400 and collimator tray 700 which can be aligned. In some implementations, the alignment elements include a groove formed in either collimator 400 or collimator tray 700 and a corresponding protrusion formed on the other component of collimation system 120. It should be appreciated that the above-mentioned alignment elements are provided as examples only and that other suitable systems for aligning collimator 400 and collimator tray 700 are contemplated herein.

### Fabrication & Customization

As mentioned above, a unique aspect of collimation system 120, e.g., for use in FLASH-RT, is that collimator 400 and/or collimator tray 700 are customizable to be suited for different use cases. For example, the size and shape of longitudinal body 402, base 404, and/or central bore 406 of collimator 400 may be adapted based on the desired size/shape of the field of irradiation. Accordingly, the size/shape of central opening 706 of collimator tray 700 may be adapted to correspond to the size/shape of longitudinal body 402 of collimator 400. For example, if longitudinal body 402 is square-shaped, then collimator tray 700 may also be fabricated with a square-shaped central opening 706, e.g., having slightly larger dimensions than longitudinal body 402 so that longitudinal body 402 can be inserted therethrough. In a similar manner, the size/shape of collimator tray 700 itself may be selected based on the attachment mechanism(s) provided on linac 100. In FIGS. 7-9, for example, collimator tray 700 is generally square-shaped to correspond to accessory rail assembly 106 of linac 100; however, collimator tray 700 may alternatively be rectangular, circular, non-uniform, etc., and the specific dimensions of collimator tray 700 are not limited.

In some implementations, collimation system 120 can be produced as a kit that includes a collimator tray (e.g., collimator tray 700) sized for a particular linac and a plurality of different collimators of different sizes/shapes that are adapted to fit with the single collimator tray. For example, a kit could include a collimator tray and a plurality of different collimators having different length bodies. In some implementations, rather than fabricating an entirely new collimator tray 700, e.g., to fit different sizes/shapes of collimator 400, various adaptors may be fabricated and/or included in a kit to adapt central opening 706 of collimator tray 700 to different sizes/shapes of collimators. For example, a variety of ring-shaped adaptors could be fabricated that have an outer diameter corresponding to central opening 706 of collimator tray 700 and different inner diameters that correspond to different sizes of collimators.

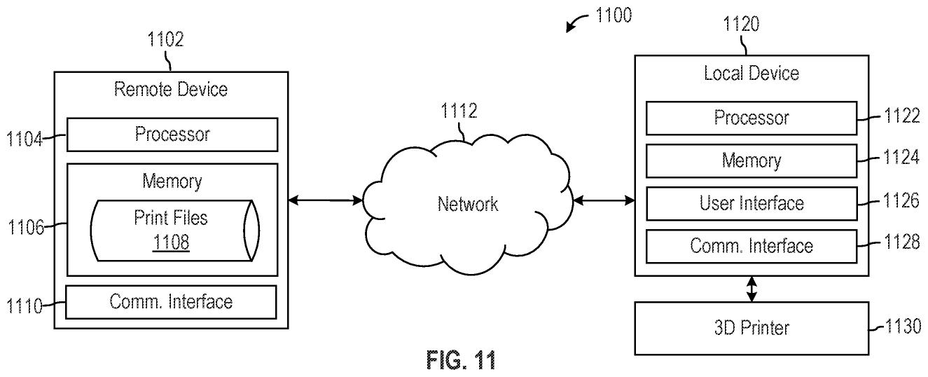

Referring now to FIG. 11, a block diagram of a system 1100 for fabricating collimators and/or trays, e.g., of different sizes/shapes-including collimator 400 and/or collimator tray 700—is shown, according to some implementations. At a high level, system 1100 includes a remote device 1102 in communication with a local device 1120 via a network 1112. Remote device 1102 is generally configured to maintain a database 1108 of 3D printing files for one or more variations of collimator 400 and/or collimator tray 700, which can be retrieved by local device 1120, modified (if desired), and used to fabricate collimator 400 and/or collimator tray 700, e.g., via a 3D printer 1130. Additional details are provided below.

Remote device 1102 is shown to include a processor 1104 and a memory 1106, which may be communicably connected via a processing circuit (not shown). Processor 1104 can be a general-purpose processor, an application-specific integrated circuit (ASIC), one or more field programmable gate arrays (FPGAs), a group of processing components (e.g., a central processing unit (CPU)), or other suitable electronic processing structures. In some implementations, processor 1104 is configured to execute program code stored on memory 1106 to cause remote device 1102 to perform one or more operations, as described below in greater detail. It will be appreciated that, in some implementations, remote device 1102 is part of another computing device (e.g., a server); accordingly, in such implementations, the components of remote device 1102 may be shared with, or the same as, the host device. For example, if remote device 1102 is implemented via a server, then remote device 1102 may utilize the processing circuit, processor(s), and/or memory of the server to perform the functions described herein.

Memory 1106 can include one or more devices (e.g., memory units, memory devices, storage devices, etc.) for storing data and/or computer code for completing and/or facilitating the various processes described in the present disclosure. In some implementations, memory 1106 includes tangible (e.g., non-transitory), computer-readable media that stores code or instructions executable by processor 1104. Tangible, computer-readable media refers to any physical media that is capable of providing data that causes remote device 1102 to operate in a particular fashion. Example tangible, computer-readable media may include, but is not limited to, volatile media, non-volatile media, removable media and non-removable media implemented in any method or technology for storage of information such as computer readable instructions, data structures, program modules or other data. Accordingly, memory 1106 can include random access memory (RAM), read-only memory (ROM), erasable programmable read-only memory (EPROM), electronically erasable programmable read-only memory (EEPROM), hard drive storage, temporary storage, non-volatile memory, flash memory, optical memory, or any other suitable memory for storing software objects and/or computer instructions. Memory 1106 can include database components, object code components, script components, or any other type of information structure for supporting the various activities and information structures described in the present disclosure. Memory 1106 can be communicably connected to processor 1104, such as via a processing circuit, and can include computer code for executing (e.g., by processor 1104) one or more processes described herein.

While shown as individual components, it will be appreciated that processor 1104 and/or memory 1106 can be implemented using a variety of different types and quantities of processors and memory. For example, processor 1104 may represent a single processing device or multiple processing devices. Similarly, memory 1106 may represent a single memory device or multiple memory devices. Additionally, in some implementations, remote device 1102 may be implemented within a single computing device (e.g., one server, one housing, etc.). In other implementations, remote device 1102 may be distributed across multiple servers or computers (e.g., that can exist in distributed locations). For example, remote device 1102 may include multiple distributed computing devices (e.g., multiple processors and/or memory devices) in communication with each other that collaborate to perform operations. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by two or more computers.

Remote device 1102 is also shown to include a communications interface 1110 that facilitates communications (e.g., the exchange of data) between remote device 1102 and any external components or devices, including local device 1120. Communications interface 1110 can be or include a wired and/or wireless communications interface (e.g., jacks, antennas, transmitters, receivers, transceivers, wire terminals, etc.) for conducting data communications, or a combination of wired and/or wireless communication interfaces. As shown, communications via communications interface 1110 may be conducted via network 1112. Network 1112 can be or include any type of communications network. For example, network 1112 may be a wide area network (WAN) (e.g., the Internet), a local area network (LAN), a virtual private network (VPN), etc. Accordingly, it should be appreciated that network 1112 may support wired or wireless communications. In some such implementations, for example, communications interface 1110 may include one or more Ethernet ports, a Wi-Fi transceiver, cellular or mobile phone communications transceivers, or other components suitable for wired or wireless communications via network 1112.

As shown, local device 1120 generally also includes a processor 1122, memory 1124, and a communications interface 1128. As with processor 1104, described above, processor 1122 can be a general-purpose processor, an ASIC, one or more FPGAs, a group of processing components (e.g., a central processing unit (CPU)), or other suitable electronic processing structures. In some implementations, processor 1122 is configured to execute program code stored on memory 1124 to cause local device 1120 to perform one or more operations. It will be appreciated that, in some implementations, local device 1120 is part of another computing device (e.g., a server); accordingly, in such implementations, the components of local device 1120 may be shared with, or the same as, the host device. For example, if local device 1120 is implemented via a server, then local device 1120 may utilize the processing circuit, processor(s), and/or memory of the server to perform the functions described herein.

Memory 1124 can include one or more devices (e.g., memory units, memory devices, storage devices, etc.) for storing data and/or computer code for completing and/or facilitating the various processes described in the present disclosure. In some implementations, memory 1124 includes tangible (e.g., non-transitory), computer-readable media that stores code or instructions executable by processor 1122. Tangible, computer-readable media refers to any physical media that is capable of providing data that causes local device 1120 to operate in a particular fashion. Example tangible, computer-readable media may include, but is not limited to, volatile media, non-volatile media, removable media and non-removable media implemented in any method or technology for storage of information such as computer readable instructions, data structures, program modules or other data.

Accordingly, memory 1124 can include RAM, ROM, EPROM, EEPROM, hard drive storage, temporary storage, non-volatile memory, flash memory, optical memory, or any other suitable memory for storing software objects and/or computer instructions. Memory 1124 can include database components, object code components, script components, or any other type of information structure for supporting the various activities and information structures described in the present disclosure. Memory 1124 can be communicably connected to processor 1122, such as via a processing circuit, and can include computer code for executing (e.g., by processor 1122) one or more processes described herein.

While shown as individual components, it will be appreciated that processor 1122 and/or memory 1124 can be implemented using a variety of different types and quantities of processors and memory. For example, processor 1122 may represent a single processing device or multiple processing devices. Similarly, memory 1124 may represent a single memory device or multiple memory devices. Additionally, in some implementations, local device 1120 may be implemented within a single computing device (e.g., one server, one housing, etc.). In other implementations, local device 1120 may be distributed across multiple servers or computers (e.g., that can exist in distributed locations). For example, local device 1120 may include multiple distributed computing devices (e.g., multiple processors and/or memory devices) in communication with each other that collaborate to perform operations. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by two or more computers.

Local device 1120 is also shown to include a communications interface 1128 that facilitates communications (e.g., the exchange of data) between local device 1120 and any external components or devices, including remote device 1102. Communications interface 1128 can be or include a wired and/or wireless communications interface (e.g., jacks, antennas, transmitters, receivers, transceivers, wire terminals, etc.) for conducting data communications, or a combination of wired and/or wireless communication interfaces. As shown, communications via communications interface 1128 may be conducted via network 1112, as described above. Accordingly, it should be appreciated that network 1128 may support wired or wireless communications. In some such implementations, for example, communications interface 1128 may include one or more Ethernet ports, a Wi-Fi transceiver, cellular or mobile phone communications transceivers, or other components suitable for wired or wireless communications via network 1112.

In some implementations, local device 1120 also includes a user interface 1126 that allows a user to interact with local device 1120. User interface 1126 generally includes a display device and a user input device. The display device is generally a screen, such as an LED or LCD screen, but could be any electronic device capable of displaying data and/or graphical user interfaces. The user input device can be any device or combination of devices that accept user inputs, such as a keyboard, a mouse, a joystick, buttons or arrow keys, a microphone, a camera, etc. For example, local device 1120 may be a personal computer (e.g., a laptop); therefore, user interface 1126 can include an LCD screen, a keyboard, and a mouse, along with other devices such as a webcam, speaker, and the like. In another example, user interface 1126 can include a touchscreen that can both display information and receive user inputs. As described below, local device 1120 can alternatively be integrated with 3D printer 1130 such that user interface 1126 includes a screen and user input devices (e.g., buttons, a touchscreen) of 3D printer 1130.

3D printer 1130 is, as per its name, a printer or “additive manufacturing device” configured to fabricate 3D objects via any of a variety of known 3D printing techniques, including but not limited to stereolithography (SLA), selective laser sintering (SLS), fused deposition modeling (FDM), and the like. 3D printer 1130 may, notably, be configured to print using one or more different materials based on the desired configuration of collimation system 120. For example, 3D printer 1130 may print in metal (e.g., copper, aluminum, or an alloy), plastic (e.g., PLA, ABS, etc.), or other suitable materials. In implementations where 3D printer 1130 is an FDM printer, for example, 3D printer 1130 may print by extruding PLA or ABS onto a print bed. It should be appreciated that 3D printer 1130 may also be configured to print using two or more materials (e.g., plastic and metal).

While only a single printer is shown in FIG. 11, it should also be appreciated that local device 1120 may be connected to multiple different types of printers (e.g., an FDM printer and a resin printer), such that collimators and/or trays can be fabricated out of different materials and/or using different techniques, and/or multiple 3D printers of the same type (e.g., so that multiple collimators and/or trays can be printed simultaneously). In some implementations, rather than a traditional 3D printer, system 1100 alternatively or additionally includes other types of computerized fabrication devices, such as a computerized lathe or mill.

In some implementations, local device 1120 communicates with 3D printer 1130 via communications interface 1128, e.g., using a wired or wireless connection. For example, local device 1120 may transmit 3D printing files (e.g., in STL format) to 3D printer 1130 for printing. In other implementations, 3D printing files may be manually transferred to 3D printer 1130, such as by loading the file(s) onto a flash drive or other portable media device via local device 1120 and then downloading the file(s) onto 3D printer 1130. In yet other implementations, local device 1120 may be part of, or integrated with, 3D printer 1130, such that 3D printing files can be directly received by 3D printer 1130, e.g., from remote device 1102, as discussed below. For example, local device 1120 may be a controller that is part of 3D printer 1130.

To begin fabricating a collimator and/or tray, a user of local device 1120 may first obtain a suitable 3D model or 3D print file of the collimator and/or tray, e.g., to be loaded onto 3D printer 1130 for printing. As used herein, a 3D model generally refers to a digital 3D model of the object to be printed (e.g., a collimator), whereas a 3D print file generally refers to a file of a 3D model that has been prepared for 3D printing. For example, those in the art will appreciate that a suitable slicing software may be used to prepare a 3D model (e.g., a CAD drawing) for 3D printing. However, it should be appreciated that the present disclosure is not intended to be limiting in this regard. Once a 3D printing file is obtained and/or generated, it may be transmitted, uploaded, or otherwise communicated to 3D printer 1130 to start fabrication. 3D printer 1130 may then operate to form the collimator and/or tray.

In some implementations, a 3D model of the desired collimator and/or tray can be generated locally, e.g., via user interface 1126 of local device 1120. For example, 3D modeling (e.g., CAD) software may be executed on local device 1120, which allows a user to generate a 3D model of the collimator and/or tray to be printed. In such implementations, slicing software may also be executed on local device 1120 to prepare the 3D model for printing. In other implementations, a 3D model or print file may be uploaded to local device 1120 from a removable storage device or remote device. In yet other implementations, 3D model or print file can be retrieved and/or otherwise obtained from database 1108 of remote device 1102. For example, as discussed herein, database 1108 is generally a database of 3D printing files (e.g., pre-sliced) or 3D models that have been previously generated, e.g., by one or more users, and that is maintained by remote device 1102 (e.g., a server).

Local device 1120 may obtain (e.g., request, retrieve, and/or receive) 3D printing files or models from remote device 1102, e.g., via network 1112. For example, the 3D printing files or models contained in database 1108 may be accessible via a web page, such that a user can navigate to said web page using a web browser, e.g., via user interface 1126, to view and download selected 3D printing files or models. In another example, 3D printing files or models can be transmitted from remote device 1102 to local device 1120, e.g., by a user of remote device 1102. For example, users of remote device 1102, local device 1120, and optionally additional computing devices may be able to share their locally generated 3D printing files or models for collimators and/or trays. In this manner, certain collimator and/or tray designs may be pre-generated for easy retrieval and printing. For example, a medical professional operating a clinical linac could easily retrieve files a predesigned collimator and/or tray for a FLASH-RT procedure and then print the collimator and/or tray, minimizing delays and lowering costs, since a custom collimator and/or tray does not need to be designed for each procedure. Additionally, in some implementations, users can modify obtained collimator and/or tray designs (e.g., using CAD software), e.g., via user interface 1126, prior to printing, which allows for some level of customization without necessarily requiring a complete redesign of the collimator and/or tray.

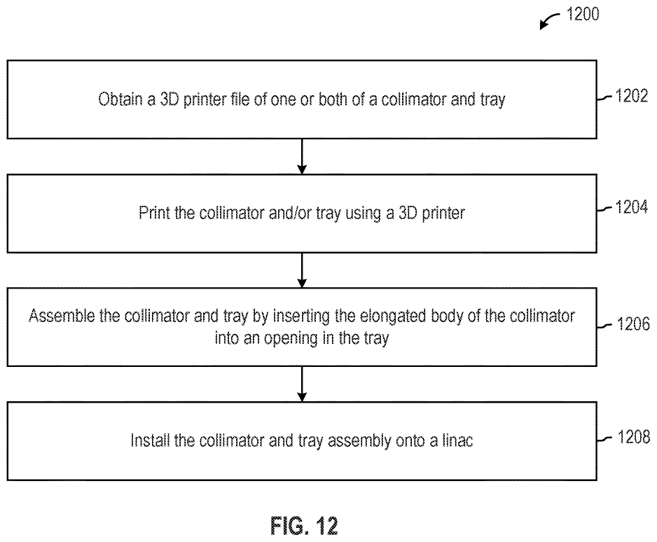

Referring now to FIG. 12, a flow chart of a process 1200 for fabricating and using a collimator and tray (e.g., collimation system 120) is shown, according to some implementations. Process 1200 can be partially implemented via system 1100, as described above, in some implementations. For example, certain steps of process 1200 may be performed by local device 1120, e.g., in conjunction with 3D printer 1130. It will be appreciated that certain steps of process 1200 may be optional and, in some implementations, process 1200 may be implemented using less than all of the steps. It will also be appreciated that the order of steps shown in FIG. 12 is not intended to be limiting.

At step 1202, 3D printer files for a collimator (e.g., collimator 400), a collimator tray (e.g., collimator tray 700), or both, are obtained. As discussed above with respect to system 1100, obtaining 3D printer file(s) for a collimator and/or tray can include retrieving said 3D printer file(s) from a remote database (e.g., database 1108), such as a database maintained by a cloud server (e.g., remote device 1102). For example, a user of a first computing device (e.g., local device 1120) may remotely access a database of 3D printer file(s), e.g., via a web browser, and may select one or more files to download onto the first computing device. Alternatively, in some implementations, 3D printer file(s) are received directly from a remote computing device. For example, a user may use a first computing device to transmit (e.g., directly, by email, etc.) 3D printer file(s) to a second computing device. In yet other implementations, 3D printer file(s) can be manually uploaded to a computing device, e.g., from removable storage, or can be generated directly on a first computing device.

At step 1204, the collimator and/or tray are printed using a 3D printer (e.g., 3D printer 1130). Accordingly, between steps 1202 and 1204, the 3D printer file(s) may be transmitted or uploaded to the 3D printer; although, as mentioned above, certain implementations are contemplated where the 3D printer obtains 3D printer file(s) directly from a database or remote device. In any case, the 3D printer may use the 3D printer file(s) to 3D print the collimator and/or tray using one or more materials. In some implementations, the collimator and/or tray are printed separately, e.g., by a single 3D printer or by multiple 3D printers. In other implementations, the collimator and/or tray can be printed together.

At step 1206, the collimator and a corresponding tray are assembled by inserting the elongated body of the collimator into an opening in the tray. As described above, the collimator generally includes a base that is wider than the elongated body such that the collimator is retained by the tray, e.g., via the base. In some implementations, assembling the collimator and tray can also include aligning corresponding alignment elements to ensure that the collimator and tray are properly assembled.

At step 1208, the collimator and tray assembly (e.g., collimation system 120) are installed on a clinical linac, e.g., for use during FLASH-RT. As mentioned above, the collimator and tray assembly may be installed onto the linac by sliding the tray onto accessory rails or other mounting elements near the exit window of the linac (e.g., as shown in FIGS. 1-3); however, other installation techniques are completed herein. For example, the collimator and tray assembly may be attached to the linac using screws, brackets, or other suitable attachment systems. Once installed, the linac can be operated to perform FLASH-RT on a subject, e.g., to treat a tumor or the like, with the output of the linac being affected by the collimator.

### Testing and Results

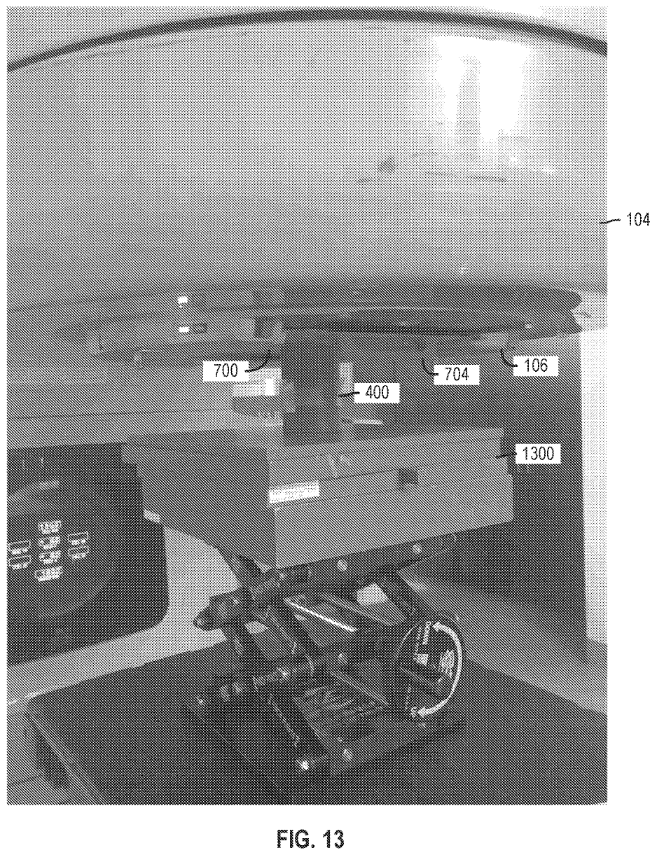

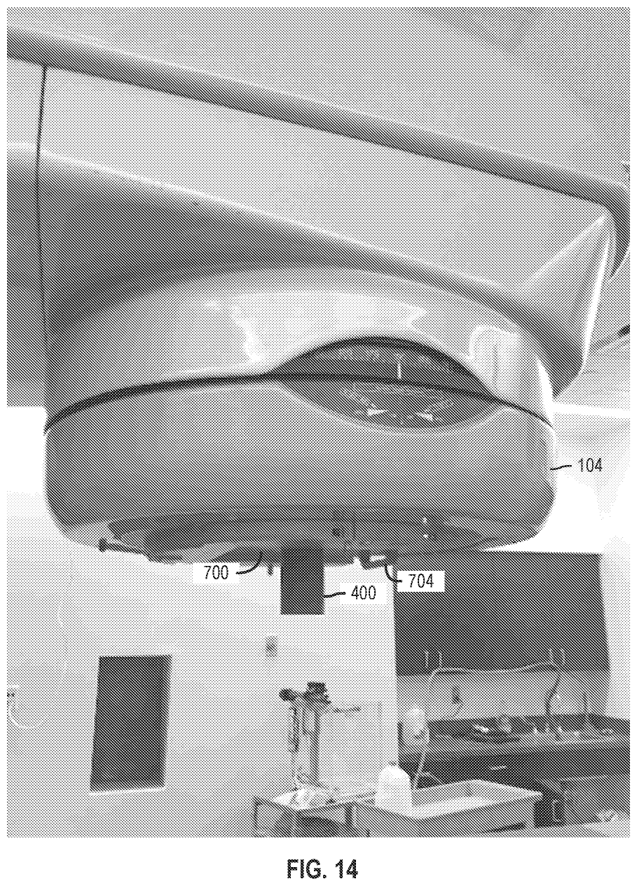

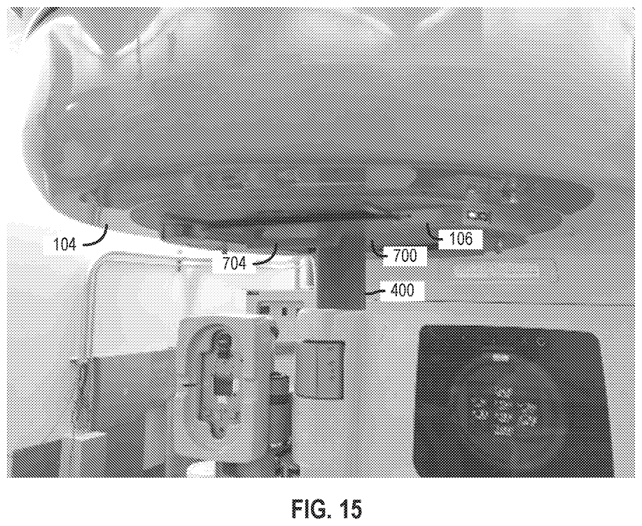

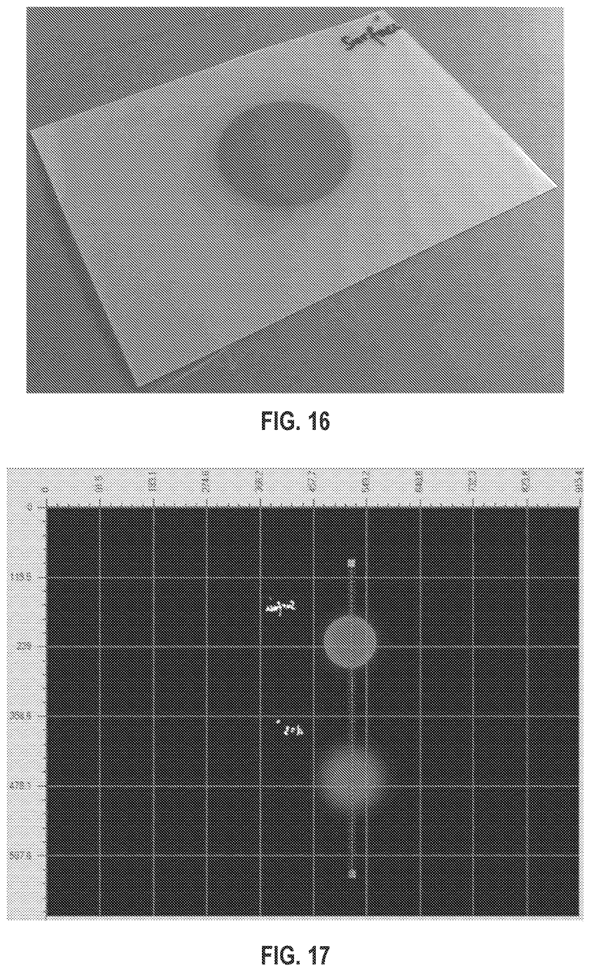

Referring now to FIGS. 13-15, various perspective views of a prototype of collimator system 120 installed on an example linac are shown, according to some implementations. In this example, the linac is a Varian® Clinac™ linac with FLEX research toolkit; although, this example is not intended to be limiting. As shown, collimator system 120 is installed onto the linac for testing. In FIGS. 13-15, in particular, a test setup is shown which includes a test stand 1300 for holding one or more sheets of radiochromic film, which are used to measure the dose and field size of radiation provided by the linac through collimator 400. In this example, test stand 1300 generally defines a treatment field below collimator 400. FIG. 16 shows an example radiotherapy dose applied to the aforementioned radiochromic film held by test stand 1300. In this example, the darkness of the film is related to the amount of dose delivered and shape of the field is determined by the collimator. Thus, as shown, collimator 400 (e.g., in the example configuration described herein) generally produces a highly concentrated, circular field.

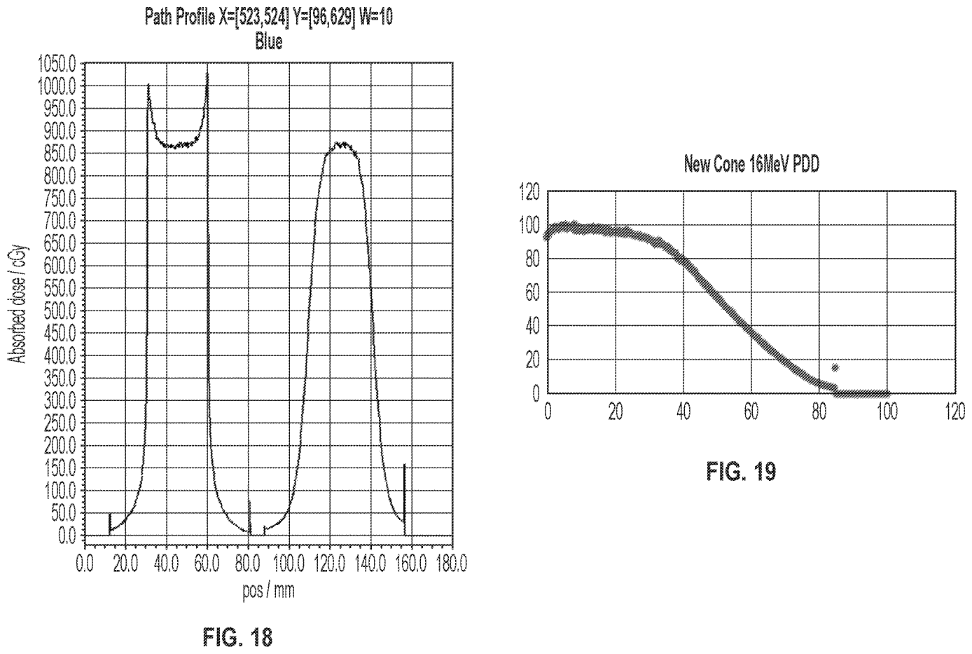

Referring now to FIGS. 17-19, the results of additional testing are shown. Specifically, FIGS. 17 and 18 are results of testing in dosimetrically water equivalent plastic. FIG. 17 shows an example diagram of a radiotherapy dose applied to a radiochromic film at the end of collimator 400 at depths of 0 cm (e.g., at the surface) and 3 cm under “water”, according to some implementations. Subsequently, the radiochromic films were scanned using analysis software to quantify the amount of dose and dose distribution (illustrated in FIG. 18). FIG. 19 shows the results of yet further testing in water. In this example, the percent depth dose curve using radiochromic film in water is quantified.

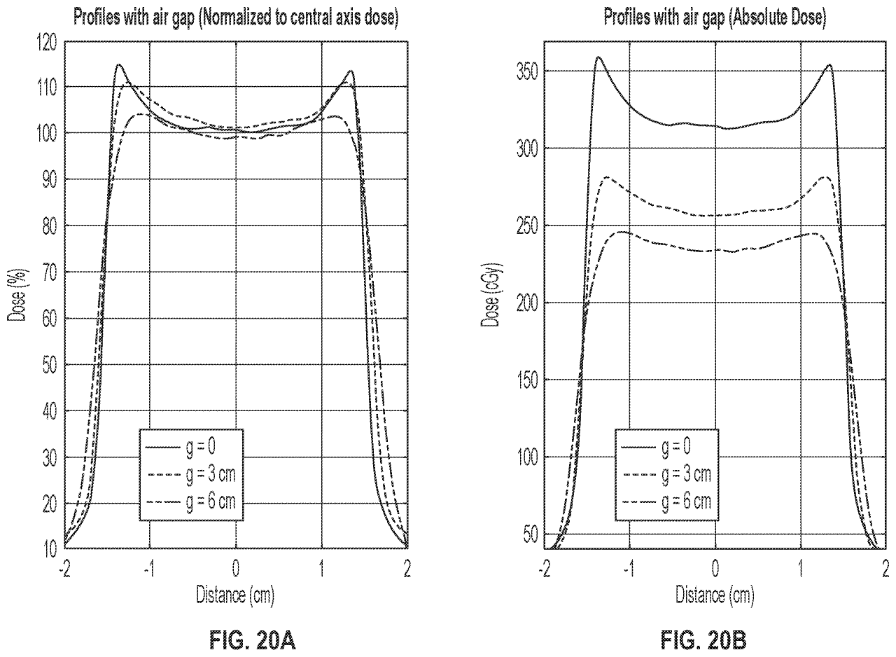

Referring now to FIGS. 20A and 20B, example dose profiles measured when testing the prototype of collimation system 120 mentioned above are shown. Specifically, FIG. 20A shows profiles with the dose normalized to the central axis for three different air gaps. FIG. 20B shows profiles with absolute dose for three different air gaps, which illustrates a decrease in dose per pulse with increasing air gap. As shown, hotspots in these profiles (e.g., where dose reaches about 115% in FIG. 20A) can be mitigated by introducing an air gap between the collimator exit window and the target tissue (or phantom). For example, FIG. 20A demonstrates that introducing a 3-cm air gap improves homogeneity to within 110% of central axis, and a 6-cm air gap improves homogeneity to within 105%. Increasing the air gap does lead to a dose per pulse decrease, e.g., with respect to the dose with no air gap, of about 80% for a 3-cm air gap and 75% for a 6-cm air gap. The field size described by the full width at half max (FWHM) is increased by 3-mm going from a 0 to 6-cm air gap.

Through testing, it was also found that activation of high-Z collimator components is observed with high-energy electron beams. Higher activation dose rates (e.g., up to three orders of magnitude higher than background) have been noted with irradiation of copper inserts with 16 MeV electrons. In contrast, with low-Z materials (e.g., PLA), dose rates measured were about two orders of magnitude lower than that measured after irradiation of higher-Z components.

### Configuration of Certain Implementations

Disclosed are components that can be used to perform the disclosed methods and systems. These and other components are disclosed herein, and it is understood that when combinations, subsets, interactions, groups, etc. of these components are disclosed that, while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific implementation or combination of implementations of the disclosed methods.

The construction and arrangement of the systems and methods as shown in the various implementations are illustrative only. Although only a few implementations have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method steps may be varied or re-sequenced according to alternative implementations. Other substitutions, modifications, changes, and omissions may be made in the design, operating conditions, and arrangement of the implementations without departing from the scope of the present disclosure.

The present disclosure contemplates methods, systems, and program products on any machine-readable media for accomplishing various operations. The implementations of the present disclosure may be implemented using existing computer processors, or by a special purpose computer processor for an appropriate system, incorporated for this or another purpose, or by a hardwired system. Implementations within the scope of the present disclosure include program products including machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures, and which can be accessed by a general purpose or special purpose computer or other machine with a processor.

When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general-purpose computer, special purpose computer, or special purpose processing machines to perform a certain function or group of functions.

Although the figures show a specific order of method steps, the order of the steps may differ from what is depicted. Also, two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule-based logic and other logic to accomplish the various connection steps, processing steps, comparison steps and decision steps.

It is to be understood that the methods and systems are not limited to specific synthetic methods, specific components, or to particular compositions. It is also to be understood that the terminology used herein is for the purpose of describing particular implementations only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another implementation includes from the one particular value and/or to the other particular value. Similarly, when values are expressed as approximations, by use of the antecedent “about,” it will be understood that the particular value forms another implementation. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal implementation. “Such as” is not used in a restrictive sense, but for explanatory purposes.

What is claimed is:

1. A collimator assembly for a linear accelerator for use in ultra-high dose rate (FLASH) radiotherapy, the collimator assembly comprising:
a collimator comprising an elongated body and a base, wherein the elongated body is cylindrically shaped, wherein the base is cylindrically shaped or disk-shaped and is positioned at one end of the elongated body, wherein the elongated body is a first diameter and the base is a second diameter that is greater than the first diameter, and wherein an opening that defines a central bore is formed in the elongated body and the base, the opening extending a length of the collimator along a central axis; and
a tray comprising a first side, a second side opposite the first side, and a central opening that extends from the first side to the second side, wherein the tray is adapted to be removably installed on accessory rails of the linear accelerator, wherein the central opening of the tray is a third diameter that greater than the first diameter of the elongated body of the collimator but smaller than the second diameter of the base of the collimator to allow only the elongated body of the collimator to pass therethrough such that the base of the collimator interfaces with the first side of the tray, thereby retaining the collimator for reversible installation on the linear accelerator.

2. The collimator assembly of claim 1, wherein the collimator is formed of a material having a density between 0.9 and 1.1 g/cm³.

3. The collimator assembly of claim 2, wherein the material is acrylonitrile butadiene styrene (ABS) or polylactic acid (PLA).

4. The collimator assembly of any of claims 1-3, wherein the collimator is formed of a copper or a copper alloy.

5. The collimator assembly of any of claims 1-3, wherein the collimator is formed of aluminum or an aluminum alloy.

6. The collimator assembly of any of claims 1-5, wherein the collimator is at least 90 millimeters (mm) in length.

7. The collimator assembly of claim 6, wherein the collimator is between 10 mm and 35 mm in length.

8. The collimator assembly of any of claims 1-7, wherein the collimator is fabricated by 3D printing.

9. The collimator assembly of any of claims 1-8, wherein the tray further comprises a handle extending from the second side of the tray.

10. The collimator assembly of claim 9, wherein the handle is integrally formed into the second side of the tray.

11. The collimator assembly of any of claims 1-10, wherein the first diameter corresponds to a desired field size for ultra-high dose rate radiation.

12. The collimator assembly of any of claims 1-11, wherein the first diameter is 3 centimeters (cm).

13. The collimator assembly of any of claims 1-12, wherein the linear accelerator is configured to generate an electron beam having an energy of at least 16 megaelectron volts (MeV).

14. The collimator assembly of any of claims 1-13, wherein the elongated body and the base are integrally formed.

15. The collimator assembly of any of claims 1-14, wherein the elongated body and the base are fabricated separately and coupled by an adhesive, welding, or soldering.

16. The collimator assembly of any of claims 1-15, wherein the opening that defines the central bore has a circular cross-section.

17. The collimator assembly of any of claims 1-16, wherein the opening that defines the central bore has a square, rectangular, or non-uniform cross-section.

18. The collimator assembly of any of claims 1-17, wherein the second diameter is 10% to 50% greater than the first diameter.

19. The collimator assembly of any of claims 1-18, wherein each of the collimator and the tray comprise alignment elements for aligning the collimator and the tray during installation.

20. A method of fabricating a collimation system for a linear accelerator, for use in ultra-high dose rate (FLASH) radiotherapy, the method comprising:
obtaining, by a first computing device, from a remote computing device, 3D printing files or 3D models of a collimator and a collimator tray, wherein the collimator tray is configured to retain the collimator for reversible installation onto the linear accelerator;
operating, by the first computing device, a 3D printer to print the collimator, wherein the collimator comprises an elongated body and a base, wherein the base is wider than the elongated body, and wherein an opening that defines a central bore is formed in the elongated body and the base, the opening extending a length of the collimator along a central axis; and
operating, by the first computing device, the 3D printer to print the collimator tray, wherein the collimator tray comprises a first side, a second side opposite the first side, and a central opening that extends from the first side to the second side, wherein the central opening is sized to allow only the elongated body of the collimator, but not the base of the collimator, to pass therethrough, and wherein the tray is adapted to be removably installed on accessory rails of the linear accelerator;
wherein the collimation system is assembled by inserting the elongated body of the collimator into the central opening of the tray such that the base of the collimator interfaces with the first side of the tray, and wherein the collimation system is installed onto the linear accelerator such that the collimator is positioned at an exit window of the linear accelerator.

21. The method of claim 20, wherein: (i) the elongated body of the collimator is cylindrically shaped, wherein the base of the collimator is cylindrically shaped or disk-shaped and is positioned at one end of the elongated body, (ii) the elongated body is a first diameter and the base is a second diameter that is greater than the first diameter, and (iii) the central opening of the tray is a third diameter that greater than the first diameter of the elongated body of the collimator but smaller than the second diameter of the base of the collimator.

22. The method of claim 20 or 21, wherein the collimator is 3D printed of a material having a density between 0.9 and 1.1 g/cm³.

23. The method of claim 22, wherein the material is acrylonitrile butadiene styrene (ABS) or polylactic acid (PLA).

24. The method of claim 20, wherein the collimator is 3D printed of a copper or a copper alloy.

25. The method of claim 20, wherein the collimator is 3D printed of aluminum or an aluminum alloy.

26. The method of any of claims 20-25, wherein the collimator is at least 90 millimeters (mm) in length.

27. The method of claim 26, wherein the collimator is between 10 mm and 35 mm in length.

28. The method of any of claims 20-27, wherein the tray further comprises a handle extending from the second side of the tray.

29. The method of claim 28, wherein the handle is integrally formed into the second side of the tray.

30. The method of any of claims 20-29, wherein the linear accelerator is configured to produce an electron beam having an energy of at least 16 megaelectron volts (MeV).

31. The method of any of claims 20-30, wherein the opening that defines the central bore has a circular cross-section.

32. The method of any of claims 20-31, wherein the opening that defines the central bore has a square, rectangular, or non-uniform cross-section.

33. The method of any of claims 20-32, wherein each of the collimator and the tray comprise alignment elements for aligning the collimator and the tray during installation.

34. A method of operating a linear accelerator configured for ultra-high dose rate (FLASH) radiotherapy, the method comprising:
providing a collimator comprising an elongated body and a base, wherein the elongated body is cylindrically shaped, wherein the base is cylindrically shaped or disk-shaped and is positioned at one end of the elongated body, wherein the elongated body is a first diameter and the base is a second diameter that is greater than the first diameter, and wherein an opening that defines a central bore is formed in the elongated body and the base, the opening extending a length of the collimator along a central axis; and
providing a tray comprising a first side, a second side opposite the first side, and a central opening that extends from the first side to the second side, wherein the tray is adapted to be removably installed on accessory rails of the linear accelerator;
inserting the elongated body of the collimator into the central opening of the tray, wherein the central opening of the tray is a third diameter that greater than the first diameter of the elongated body of the collimator but smaller than the second diameter of the based on the collimator to allow only the elongated body of the collimator to pass therethrough such that the base of the collimator interfaces with the first side of the tray; and
installing the tray on accessory rails of the linear accelerator such that the collimator is positioned at an exit window of the linear accelerator.

35. The method of claim 34, wherein the collimator is formed of a material having a density between 0.9 and 1.1 g/cm³.

36. The method of claim 35, wherein the material is acrylonitrile butadiene styrene (ABS) or polylactic acid (PLA).

37. The method of claim 34, wherein the collimator is formed of a copper or a copper alloy.

38. The method of claim 34, wherein the collimator is formed of aluminum or an aluminum alloy.

39. The method of any of claims 34-38, wherein the collimator is at least 90 millimeters (mm) in length.

40. The method of claim 39, wherein the collimator is between 10 mm and 35 mm in length.

41. The method of any of claims 34-40, wherein the collimator is fabricated by 3D printing.

42. The method of any of claims 34-41, wherein the tray further comprises a handle extending from the second side of the tray.

43. The method of claim 42, wherein the handle is integrally formed into the second side of the tray.

44. The method of any of claims 34-43, wherein the first diameter corresponds to a desired field size for ultra-high dose rate radiation.

45. The method of any of claims 34-44, wherein the first diameter is 3 centimeters (cm).

46. The method of any of claims 34-45, wherein the linear accelerator is configured to produce an electron beam having an energy of at least 16 megaelectron volts (MeV).

47. The method of any of claims 34-46, wherein the elongated body and the base are integrally formed.

48. The method of any of claims 34-47, wherein the elongated body and the base are fabricated separately and coupled by an adhesive, welding, or soldering.

49. The method of any of claims 34-48, wherein the opening that defines the central bore has a circular cross-section.

50. The method of any of claims 34-49, wherein the opening that defines the central bore has a square, rectangular, or non-uniform cross-section.

51. The method of any of claims 34-50, wherein the second diameter is 10% to 50% greater than the first diameter.

52. The method of any of claims 34-51, wherein each of the collimator and the tray comprise alignment elements for aligning the collimator and the tray during installation.
